(19) United States
(12) Patent Application Publication
(10) Pub. No.: US 2009/0011955 A1
(43) Pub. Date: Jan. 8, 2009

(54) Method for Localization of Nucleic Acid Associated Molecules and Modifications

(75) Inventors: Piero Mariano, Strasbourg (FR); Rolf Ohlsson, Uppsala (SE)

Correspondence Address: FOLEY & LARDNER LLP, 111 HUNTINGTON AVENUE, 26TH FLOOR, BOSTON, MA, 02199-7610, US

(73) Assignee: Forskarpatent l Uppsala AB

(21) Appl. No.: 11/994,658

(22) PCT Filed: Jul. 3, 2006
(86) PCT No.: PCT/SE2006/050239

§ 371 Date: Sep. 11, 2008

(30) Foreign Application Priority Data
Jul. 6, 2005 (SE) 0501606-8

(51) Int. Cl. C40B 40/06; C12Q 1/68; C40B 50/06

(52) U.S. Cl. 506/16; 435/6; 506/26

(57) ABSTRACT

The present invention relates to a method for the localization of at least one molecule associated with, or site of interest in, a sample nucleic acid, comprising the steps—Bringing at least one reporter complex, comprising at least one binding part showing specific binding to the molecule or the site of interest and at least one reporter nucleic acid, into contact with the sample nucleic acid,—Fragmenting the sample nucleic acid,—Enzymatically ligating the reporter complex nucleic acid(s) to the sample nucleic acid, and—Detecting the hybrid ligation product. The invention also relates to libraries made with the method as well as microarrays of such libraries.

### BACKGROUND OF THE INVENTION

Essentially all the biological functions of nucleic acids are realized and regulated by their direct or indirect interaction with other molecules at specific locations and by the modifications to which a nucleic acid can be subjected. The complexity of such interactions and modifications is particularly remarkable in higher organisms. Cells within developing multicellular eukaryotes, for example, build tissue-specific chromatin architectures to express certain genes and silence others.

In the past three decades, since the nucleosome model of chromatin emerged, there has been considerable progress in elucidating how that structure and its various epigenetic states contribute to the regulatory process. One example is given by the widely accepted concept of the “histone code” where the distinct qualities of a certain chromatin region are dependent on the local concentration of modified nucleosomes that will permit the assembly of different epigenetic states leading to distinct interpretation of the genetic information (expression, repression or proliferation, differentiation).

An additional level of complexity is added by the combinatorial effect of different modifications occurring in the same region. Emerging evidence shows for example that the simultaneous presence of different nucleosome post-translational modifications can be required to recruit a specific factor to a particular region.

From this perspective the sensitive and precise mapping of these interactions and modifications, both at a sequence-specific and at genome wide level, constitutes one of the major challenges in the post genomic era.

Much of the advance in the field is due of course to the new tools of molecular biology; in particular one technique has revolutionized the study of protein-DNA interactions in vivo, the chromatin immunoprecipitation or ChIP assay (for a review, see e.g. Das et alⁱ) ⁱ Das, P. M. et al, BioTechniques, vol 37, No. 6, 2004, 961-969

The ChIP assay is a widely used method for the analysis of the association of specific proteins, or of their modified isoforms, with defined genomic regions. Depending on the protein to be analysed there are two main variants of the ChIP assay that basically differs only in the preparation of the starting chromatin.

The first uses native chromatin (thereby named NChIP) that is prepared by micrococcal nuclease digestion of the nuclei. This approach is suitable mainly for histones and their modified forms or for very strong DNA binding factors. In all other cases a second approach is preferred where the chromatin is crosslinked (thereby named XChIP) generally with formaldehyde and then fragmented by sonication. In both cases the final immunoprecipitate is analysed by PCR amplification. Recently two new techniques have been developed (ChIC, chromatin immunocleavage, and ChEC, chromatin endogenous cleavage) where the antibodies are coupled to micrococcal nucleases and the interaction of a specific factor with a particular region is revealed by the presence of a hypersensitive site.

There are several drawbacks of the ChIP, ChIC and ChEC assays. Micrograms of DNA (chromatin) are required for each assay, and this is a severe limitation when rare biological samples (like biopsies, early embryonic stages etc) are investigated, or when the same analysis has to be performed for more than one factor.

It is not possible to assess more than one factor in the same sample (test tube). This can of course be done separately, but would then require the double amount of starting material.

It is difficult to assess if two or more factors are simultaneously located on the same region and practically impossible if the amount of sample is limited as this would require two or more sequential immunoprecipitations. This is a serious limitation in the study of combinatorial effects of different factors/modifications.

The XChIP assay has a relatively poor resolution. This is a consequence of the sonication step used in current XChIP protocols that results in a main population of fragments with a defined average size, but with a significant degree of subpopulations of fragments of greater and smaller size compared to the average. As a consequence it is very difficult, if not impossible, to discriminate between two sites separated only a few hundred base pair using an ordinary ChIP.

Dekker et alⁱⁱ has described the 3C-methodology (Chromosome Conformation Capture), wherein intact nuclei are isolated and subjected to formaldehyde fixation which cross-links touching proteins and DNA. The DNA is digested by EcoRI, cross-linked fragments are ligated and amplified by PCR. This method is thus only suitable for the study of protein mediated DNA-DNA interactions. ⁱⁱ Dekker, J. et al, Science, vol 295, 2002, 1306-1309

Further technologies involving specific protein-protein or protein-DNA interactions and PCR are Immuno-PCRⁱⁱⁱ and antibody-based proximity ligation^{iv}. ⁱⁱⁱ Niemeyer, C. M. et al, TRENDS Biotechnol, vol 23, No 4, 2005, 208-216^{iv} Gullberg, M. et al, PNAS, vol 101, No 22, 2004, 8420-8424

There is consequently a need for an improved method for the sensitive high throughput analysis of in vivo and in vitro protein-nucleic acid interactions and of the modifications.

### SUMMARY OF THE INVENTION

The present invention relates in a first aspect to a method for the localization of at least one molecule associated with, or site of interest in, a sample nucleic acid, comprising the steps
Bringing at least one reporter complex, comprising at least one binding part showing specific binding to the molecule or the site of interest and at least one reporter nucleic acid, into contact with the sample nucleic acid,
Fragmenting the sample nucleic acid,
Enzymatically ligating the reporter complex nucleic acid(s) to the sample nucleic acid, and
Detecting the hybrid ligation product.

In a second aspect the invention relates to a method for producing a library of fragments of interest from a sample nucleic acid, comprising the steps
Bringing at least one reporter complex, comprising at least one binding part, showing specific binding to a nucleic acid binding factor or a site of interest in the sample nucleic acid, and at least one reporter nucleic acid, into contact with the sample nucleic acid,
Fragmenting the sample nucleic acid,
Enzymatically ligating the reporter nucleic acid to the sample nucleic acid,
Amplifying the ligated reporter/sample nucleic acid.

This aspect further includes the libraries obtained or obtainable by the method and solid supports having such libraries immobilized on them.

In a third aspect the invention relates to a kit comprising means for performing the methods according to the first and second aspects, such as reporter complex, ligases, buffers and instructions.

### DESCRIPTION OF THE DRAWINGS

FIG. 1 illustrates the embodiment of the invention as shown in Example A1.

FIGS. 2A and 2B illustrate the embodiment of the invention as shown in Example A2.

FIG. 3 illustrates the embodiment of the invention as shown in Example A3.

FIG. 4 illustrates the embodiment of the invention as shown in Example D2.

FIG. 5 illustrates the embodiment of the invention as shown in Example F.

### DETAILED DESCRIPTION OF THE INVENTION

This invention relates to the detection and localization of binding proteins or binding sites on nucleic acids, e.g. chromatin associated factors, without the drawbacks of existing methods. This is accomplished by a number of steps. The first step is to construct a reporter complex. This reporter complex comprises one part that is a protein, polypeptide, peptide, nucleic acid or any other molecule (reporter complex binding part) with specific binding affinity to the molecule or binding site to be localized or analysed and one part that is a nucleic acid (reporter complex nucleic acid).

The reporter complex binding part is chosen for its capability to bind to the protein or site of interest. If a protein binding to the nucleic acid is to be studied, the reporter complex binding part could i.a. be an antibody binding to said protein, or an antibody fragment with the relevant binding properties or a secondary antibody recognizing a primary antibody directed against the said protein. If a binding site on the nucleic acid is to be studied, the reporter complex binding part could i.a. be a protein, or fragment thereof, which normally binds or is suspected to bind to the binding site. The reporter complex may contain more than one binding part in order to increase the probability of binding to the site of interest.

The nucleic acid is designed to serve as a means for detecting the sample nucleic acid. It should thus have an end that can be ligated to the sample nucleic acid. If the detection is performed by PCR, the reporter complex nucleic acid should be long enough to accommodate a PCR-primer. The reporter complex nucleic acid can be DNA or RNA and double-stranded or single-stranded, depending on the application. The length of the reporter nucleic acid also depends on the application. If the sample nucleic acid fragments are 200 to 300 bases or base pairs, the reporter complex nucleic acid is preferably 50-150 bases or base pairs. If the sample nucleic acid fragments are longer, also the reporter complex nucleic acid needs to be longer. In the currently contemplated embodiments, reporter complex nucleic acids of up to around 1000 or 2000 bases or base pairs may be useful. To increase the local concentration of reporter complex nucleic acid at the site of interest, a reporter complex may comprise a number of nucleic acid molecules.

The reporter complex nucleic acid is attached to the reporter complex binding part. It may be attached in any of a number of ways, e.g. covalent bonds, streptavidin-biotin interactions or any other suitable way that makes the complex stable under the conditions it is used.

The sample comprising the sample nucleic acid is preferably purified prior to analysis. This may be done by isolating nuclei from the cells, lysis in lysis buffer and a short sonication followed by a change of buffer to facilitate digestion with restriction enzymes.

The sample nucleic acid is fragmented to convenient size. This may be done by, for example, sonication, digestion with suitable restriction enzyme(s) or, in the case the sample nucleic acid is RNA, by annealing ssDNA oligonucleotides and an enzyme that cuts DNA/RNA-hybrids, such as RnaseH.

If restriction enzymes are used, the choice of restriction enzyme(s) is done based on desired resolution of the assay. If high resolution is desired, it is preferred to use a frequent cutter (4-cutter) that yields fragments of on average 250 base pairs. If larger regions should be analysed, 6-cutters or even 8-cutters may be used. Care should also be taken to choose a restriction enzyme that is able to digest the sample nucleic acid, which may be associated with different proteins, e.g histones. Fragmentation by sonication may also be adapted to yield the desired average fragment length. When using RnaseH for fragmentation, the average size can be adjusted by designing ssDNA-oligonucleotides that bind at a certain frequency in the sample. Also, ssDNA-oligonucleotides may be designed to flank a specific sequence of interest.

The reporter complex is brought into contact with the sample nucleic acid in a suitable buffer and allowed to bind to the site of interest.

The ends of the reporter complex and sample nucleic acids are then ligated. This is done under sufficiently diluted conditions to favour intra-molecular ligation of the sample and reporter complex nucleic acids over random fragments. If the nucleic acids are dsDNA, T4 ligase can be used to ligate the nucleic acids. If the nucleic acids are ssDNA or RNA, an RNA ligase is preferred. However, any suitable ligase that can ligate the nucleic acids may be used.

Fragmentation and ligation could also be performed simultaneously. In this case the reporter complex nucleic acid would be modified so that the ligated product can not be redigested once it is formed. Alternatively, the ligation of the reporter complex nucleic acid to the sample nucleic acid would generate DNA sites that are not recognizable by the enzyme/s used to fragment the sample nucleic acid.

The ligation product is then detected. This may be done in a number of ways. Presently preferred is a PCR method wherein primers are annealed to the reporter complex nucleic acid and/or the sample nucleic acid and the PCR performed. This is followed by analysis of the thus amplified fragments, e.g. by sequencing or hybridisation to complementary probes. The PCR step may be exchanged for some other amplification method, such as qPCR, RCA. The amplified fragments may also be used as a library.

Further, more specific, embodiments are described in the following examples. These examples are only provided as illustrations of the generic method described above and defined in the appended claims.

### EXAMPLES

### Example A

### Analysis Of A Protein Factor Or A Post-Translational Modification Of A Factor

This example is explained with the reporter complex binding part being an antibody, but it is equally applicable to other reporter complex binding parts, such as antibody fragments, binding factors or fragments of such factors or other proteins/polypeptides/peptides or nucleic acids with the desired binding properties or binding properties to be investigated.

The reporter complex nucleic acid is a dsDNA.

### Example A1

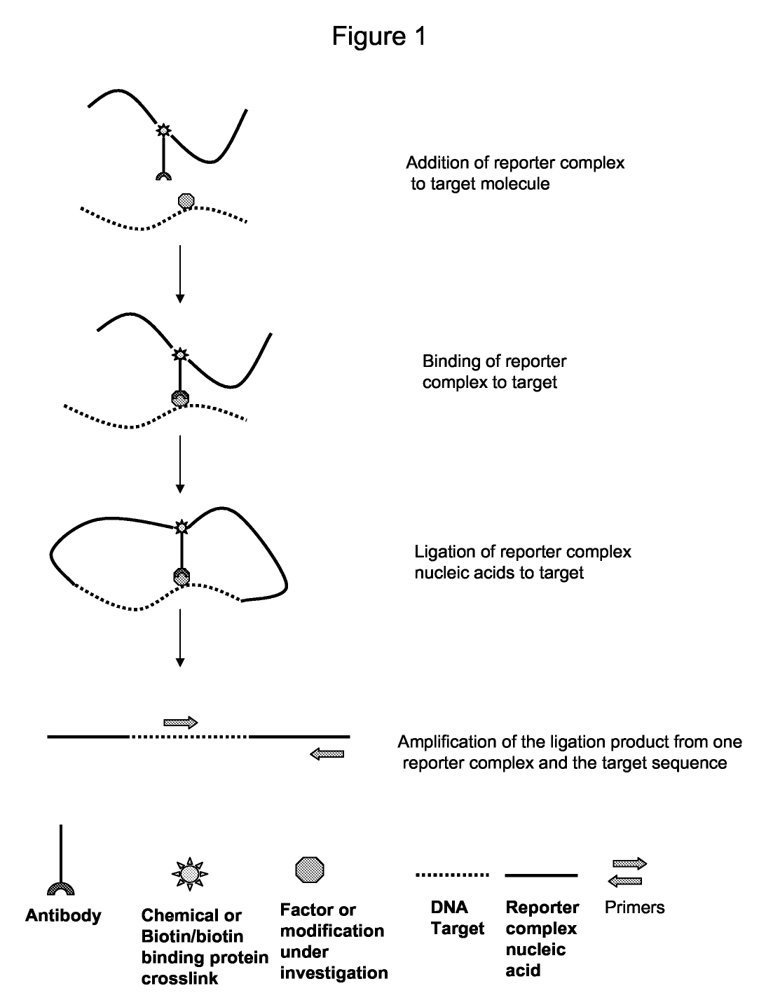

### Localization Of The Interaction Of A Factor (Or One Of Its Isoforms) Or Of A Modification At A Specific Genomic Site (FIG. 1)

In this case the assay can be performed in vivo using native or crosslinked chromatin templates, or in vitro using a naked genomic DNA template to which a factor of interest has been added.

As shown in FIG. 1 the assay is performed by fragmenting the genomic template, e.g. with an appropriate restriction enzyme, and adding the reporter complex equipped with ends compatible with those generated by the restriction enzyme on the genomic template. Thereafter, the ligation product is amplified with one primer in the reporter complex nucleic acid and another primer in the sequence under investigation

### Example A2

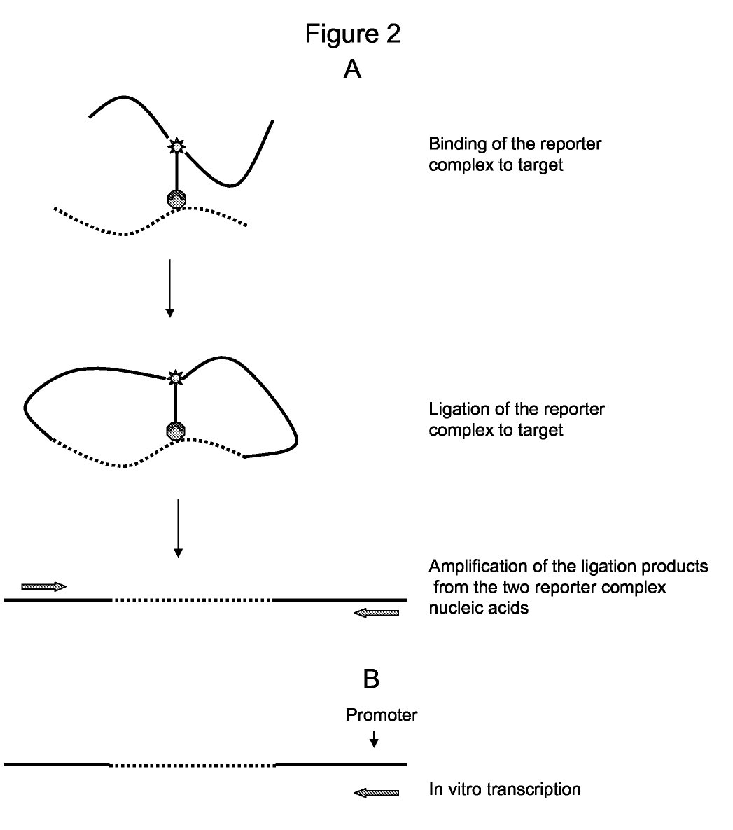

### Analysis Of The Genome-Wide Localization Of A Particular Factor (FIG. 2A and 2B)

The reporter complexes are multivalent (consequently equipped with more than one nucleic acid per reporter complex). This permits the capture of the restriction fragments generated by the restriction enzyme via binding of the reporter complex on both sides of the fragments. The assay is performed as in FIG. 1 with the difference that the final amplification of the ligation products is performed with primers both of which hybridise with the reporter complex nucleic acid (FIG. 2A).

This will result in the generation of a library of fragments representative of the genome wide localization of the factor under investigation. This library can subsequently be hybridised to microarrays with genome wide coverage and compared or hybridised to those generated with reporter complexes against other factors

A variant of this example is that the reporter complex nucleic acid is designed to contain a suitable promoter (such as the T7 promoter) for in vitro transcription. A library of fragments representative of the genome wide localization of the fragments under investigation is consequently obtained by in vitro transcription (FIG. 2B) .

### Example A3

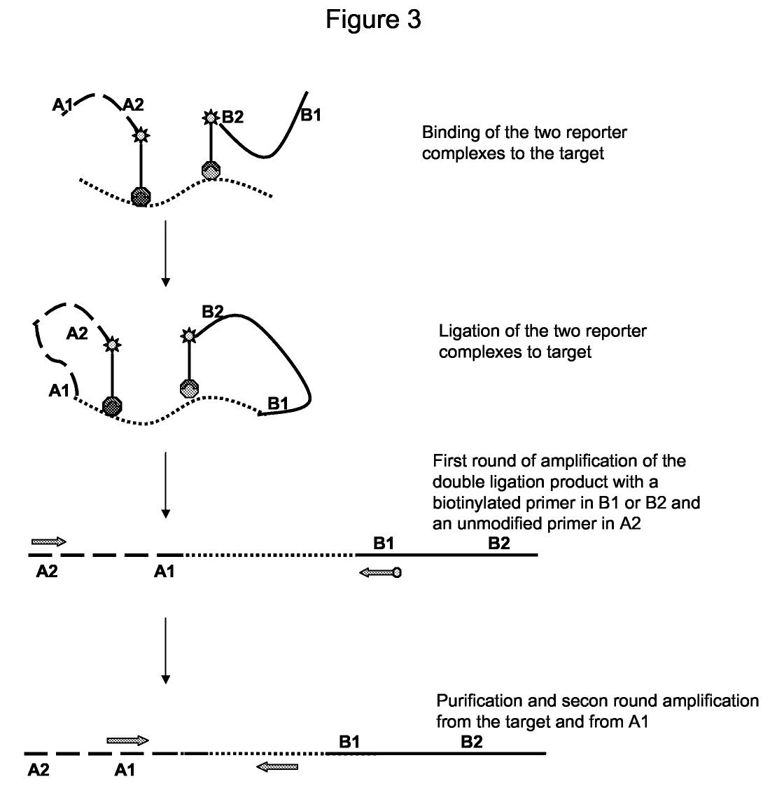

### Colocalization Of Two Factors At A Specific Genomic Region (FIG. 3)

As discussed earlier, one limitation of existing methods for the study of chromatin associated factors is the difficulty to perform sequential precipitations, due to the relative low recovery of the first step.

This is not the case in the assay according to the present invention. One advantage in having a reporter complex nucleic acid linked to the reporter complex binding part is that two or more different binding parts can be linked; each one with a specific nucleic acid allowing the detection of the different possible specific double ligation products. The advantage thus resides in the fact that all the different combinations could be assessed in the same experiment in the same test tube.

Considering the example shown in FIG. 3: Reporter complex against factor A and reporter complex against factor B are added together to the sample. The two reporter complexes differ in their nucleic acid sequences in that the reporter complex specific for factor A has a reporter complex nucleic acid comprising two primer sites A1 and A2, and the reporter complex specific for factor B analogously has the primer sites B1 and B2. The assay is carried out as described in the previous examples.

An artifact of this ligation is that the identical reporter complex nucleic acids may be ligated to both ends of the sample nucleic acid, giving the product A2A1-DNA-B1B2 (desired), but also A2A1-DNA-A1A2 and B2B1-DNA-B1B2, which are undesired. Therefore a first round of amplification is performed using a biotinylated primer that anneals to B1 (or B2) in the first reporter complex and an unmodified primer that anneals to A2 in the second reporter complex. The product of this first amplification is affinity purified through a biotin-binding protein, eluted and subjected to a second round of amplification with one primer annealing to the genomic region under analysis and a second primer annealing to A1 in the reporter complex against factor A.

A variant of this example is that the reporter complex nucleic acids A and B are designed so that the products A2A1-DNA-B1B2, A2A1-DNA-A1A2 and B2B1-DNA-B1B2 differ significantly in size. This could be done, for instance, by making A1 and A2 significantly shorter than B1 and B2 and placing the primer sequence in the B1/B2-end closest to the reporter complex binding part. This would make the A2A1-DNA-A1A2-product short, A2A1-DNA-B1B2 intermediate and B2B1-DNA-B1B2 long. The biotin affinity purification step may then be replaced by a size-dependent purification, e.g. by gel electrophoresis.

A second variant of this example is that one of the two reporter complex nucleic acid is equipped with a suitable promoter for in vitro transcription. In this case the double ligation product is in vitro transcribed from the first reporter complex nucleic acid. Subsequently the transcript is reverse transcribed with primers specific to the second reporter complex nucleic acid and the DNA sequence under investigation.

Obviously, if more than two different reporter complexes are added to the same sample, different colocalization combinations and the resulting double ligation products can be assessed in the same experiment

### Example A4

### Generation Of Libraries Of Genomic Colocalization Of Two Factors

The double ligation products, generated from two different reporter complexes and described in example A3, can provide the templates for the amplification and production of libraries of fragments representative of the genome wide colocalization of two factors.

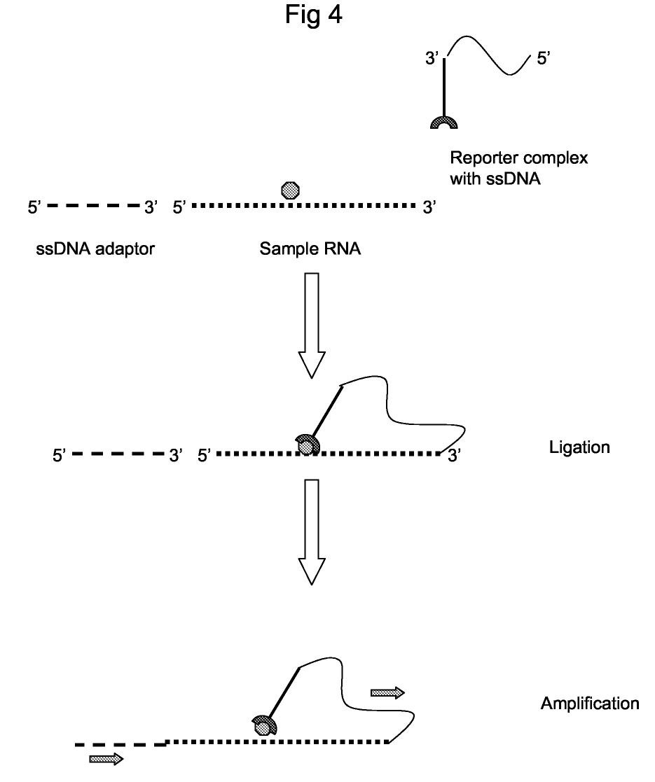

Referring to FIG. 4A, the ligation products are subjected to a first amplification round with one biotinylated primer annealing to B2 and an unmodified primer annealing in A2. The amplification products are affinity purified with a biotin-binding support, eluted and subjected to a second round of amplification with a biotinylated primer annealing in A1 and an unmodified primer annealing in B1. The affinity purification of the final amplification product provides the colocalization library.

A variant of this example is that one of the two reporter complex nucleic acids in one of the two complexes is equipped with a suitable promoter for in vitro transcription. In this case the double ligation product is in vitro transcribed from the first reporter complex nucleic acid. Subsequently the transcript is reverse transcribed with primers specific to the reporter complex nucleic acid A and B

### Single Tube Procedure

The assays described in examples A1-A4 could be performed according to the following procedure, with minimal handling in a single test tube:
Cell lysis and recovery of the nuclei by centrifugation
Resuspension in a suitable restriction/ligation buffer
Removal of uncrosslinked proteins
Release of chromatin by short sonication/vortexing
Restriction-immunoligation reaction (in this step restriction enzyme(s), ligase(s) and reporter complex(es) are added simultaneously to the sample).
De-crosslinking and DNA purification
Analysis of the immunoligated material

As in the case of the site specific approach described in example A3, the simultaneous addition of several different reporter complexes will allow the generation of the colocalization libraries (in pairs) for all the different combinations of factor analysed.

Summary of examples A1-A4
From the examples described in A1 to A4 it results that following information could be obtained from the same test tube in one experiment:
if a particular factor interacts with a specific genomic location; if a second factor interacts with the same or with another genomic location; if a third factor interacts with the same or with different locations and so on
the genome wide distribution of a specific factor; the genome wide distribution of a second factor; the genome wide distribution of a third factor and so on
if a pair of factors colocalize at a specific genomic sites; which pairs of factors among all those analysed colocalize at a specific genomic sites
the degree of genome wide colocalization of a pair of factors; the degree of genome wide colocalization of all of the factors analysed

### Example A5

### Use Of Reporter Complexes In Terminal Transferase Dependent PCR For In Vivo UV Photofootprinting

Although the use of reporter complexes as described in example A1 (as is the case for other current methods) shows a high resolution, it fails to identify the exact sequence to which a factor is bound. Such levels of single nucleotide resolution are achieved in vivo only combining ChIP assays and DNA in vivo footprinting^{v}. Obviously, these methods suffer from all the limitations already described for the ChIP assay. ^{v} Kang, S. H, et al, Nucleic Acids Res, vol 10, No 10, 2003, e44

The use of reporter complexes could be easily adapted to reveal the lesions generated by footprinting agents on genomic fragments bound by a particular factor of interest.

Terminal transferase dependent PCR (TDPCR) is for example a sensitive method for in vivo footprinting^{vi} that can be combined with the use of reporter complexes. In this embodiment of the invention, the first step of primer extension will be carried out with a biotinylated primer from the reporter complex nucleic acid. The amplified material is then isolated via streptavidin beads. Successively the material is subjected to another primer extension, but this time with a nested primer that extends into the genomic region of interest. The subsequent steps are those used in a normal TDPCR. ^{vi} Komura, J. and Riggs, A. D., Nucleic Acids res, vol 26, No 7, 1998, 1807-1811

### Examples B

### Analysis Of Covalent Modifications Of DNA

DNA molecules are subjected to different modifications (e.g. methylation of the bases). These modifications play a role in many different biological processes (gene regulation, recombination, repair etc). In this embodiment the reporter complex binding part is directed against covalent modifications of DNA and the nucleic acid is a dsDNA.

### Example B1

### Analysis Of The CpG Methylation Status At A Given Locus

The reporter complex is designed to bind to 5-methylcytosine and is therefore added to purified naked DNA. The procedure is otherwise the same as for example A1, including the digestion and ligation moments and the final PCR evaluation using one primer from the endogenous DNA site and the other from the exogenous reporter complex nucleic acid.

### Example B2

### Analysis Of The CpG Methylation On Genome Wide Scale

The analysis is performed as in A1-A4, but the reporter complex binding part is designed to bind to 5-methylcytosine

### Examples C

### Utilization Of Reporter Complexes In Targeting DNA Modifications: Methylation Analysis Of Reporter Complex Ligated Samples

### Example C1

### Methylation Analysis Of Samples Generated As In A1

After the ligation step described in example A1 the sample is reconcentrated and subjected to bisulphite mutagenesis. Finally it is amplified with one primer annealing in the reporter complex nucleic acid and a second annealing in the specific genomic region analysed. The subsequent steps are those of ordinary bisulphite sequencing. As in example A1 the analysis can be extended to samples generated by different reporter complexes in the same test tube

### Example C2

### Methylation Analysis Of Samples Generated As In A3

After the ligation step described in example A3 the sample is reconcentrated and subjected to bisulphite mutagenesis. The subsequent steps are identical to those described in A3 and the products of the second amplification are processed as in an ordinary bisulphite sequencing. As in example A3 when more than two different reporter complexes are added to the same sample, different colocalization combinations can be analysed for methylation in the same test tube.

### Examples D

### The Reporter Complex Nucleic Acid Is A ssDNA Oligonucleotide. The Sample Nucleic Acid Is An RNA

In the same way as for protein-DNA interactions, the method can be adapted for the study of protein-RNA interactions or RNA modifications or indirect DNA/RNA interactions. The reporter complex nucleic acid will be an ssDNA. The ligation is carried out by an RNA ligase.

### Example D1

### Detection Of The Association Of A Factor To A Specific RNA

A reporter complex is added to a crosslinked RNA preparation (where proteins and nucleic acids have been crosslinked by formaldehyde, UV light or any other method) that has been fragmented to a convenient average size, by for example sonication or selective digestion by RnaseH through the addition of strategically designed DNA oligonucleotides annealing on both sides of the RNA fragment to be targeted.

Ligation is performed under diluted conditions by an RNA ligase, such as T4 RNA ligase. The ligated material is purified and amplified by reverse transcription. The resulting cDNA is amplified from a primer annealing in the reporter complex nucleic acid and from a second primer annealing in the sample RNA under investigation.

### Example D2

### Generation Of A Library For The Investigation Of The Interactions Of A Factor At The Transcriptome Level

This example is illustrated in FIG. 4

Parts or all of the total RNA is fragmented to a desired average size. The reporter complex is added and subjected to a ligation reaction by an RNA ligase. Since the reporter complex nucleic acid has one of its ends conjugated to the reporter complex binding part, only one end of the reporter complex nucleic acid is available in the ligation reaction operated by the RNA ligase.

After the ligation of the reporter complex an excess of a ssDNA adaptor (that differs in sequence from the reporter complex nucleic acid) is added to ligate to the available end on the sample RNA fragments. The double ligation products can then be amplified with one primer annealing in the reporter complex and a second primer annealing in the adaptor

### Example D3

### Detection Of The Colocalization Of Two Factors On A Specific RNA Sequence

As in example D1 but with the difference that a second reporter complex for a second factor is added. The two reporter complex nucleic acids will have opposite polarity with respect to their conjugation to the antibody, i.e. one is conjugated to the reporter complex binding part at its 5′-end and one at its 3′-end. This will allow their binding to the two ends of the target RNA.

In analogy with example A3, once the double ligation product is obtained and reverse transcribed, a first PCR round is performed one primer annealing in one of the reporter complex nucleic acids and a second primer annealing in the other reporter complex nucleic acid. Finally the RNA target specificity is assessed by a second round of amplification with one primer in the RNA (now cDNA) molecule and a second primer in one of the reporter complex nucleic acid and/or, separately, in the other reporter complex nucleic acid.

### Example D4

### Generation Of A Library For The Colocalization Of Two Factors At Transcriptome Level

A library for the colocalization of two factors at transcriptome level can be obtained as in example D3 but in case the total RNA has to be fragmented to a desired average size by RnaseH this will be achieved as in example D2. A first PCR amplification from the two reporter complex nucleic acids is sufficient to generate the library

### Examples E

### Study Of RNA Modifications

The methods illustrated in Example D may easily be adapted to study of RNA modifications by using, as binding part in the reporter complex binding part, a protein (or a fragment thereof which binds, or is suspected to bind, the RNA modification of interest. The sample RNA can be a purified naked RNA.

### Example F

### 3D Conformation Studies

The development of two new techniques, the “3C” and the “RNA trap assay” have enabled the visualization of the spatial organization of chromosomal regions at a resolution level not possible just a few years ago. However these methods do not provide any information about the possible proteins involved in those three-dimensional structures.

The utilization of the reporter complexes according to the invention facilitates the prediction of the factorology involved in setting up and maintaining these three-dimentional structures by assessing the presence of a given factor inside such complexes.

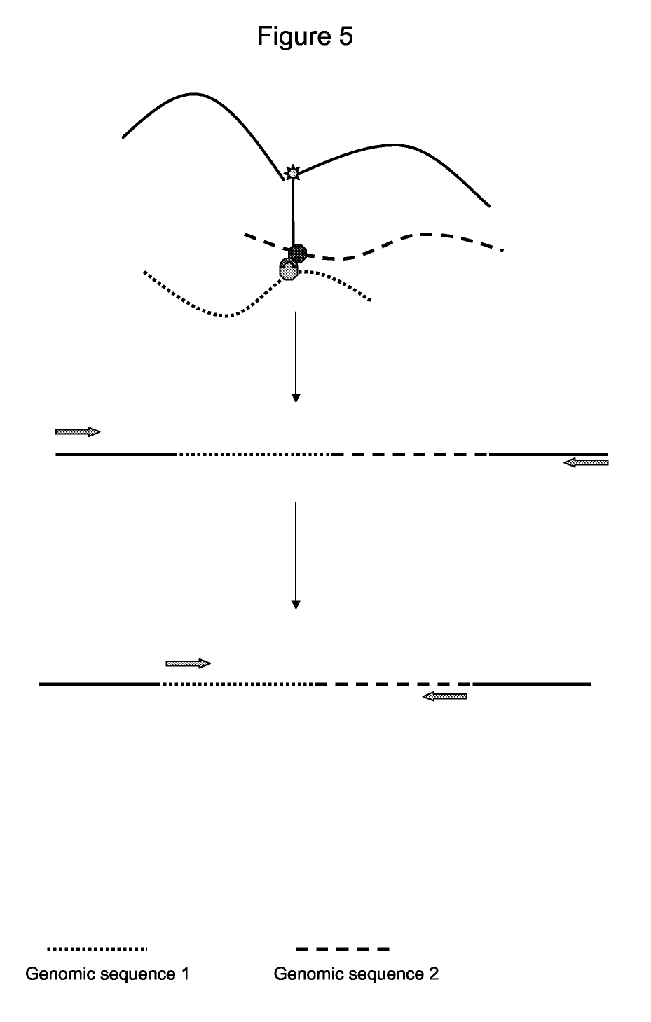

This adaptation is based on the assumption that when two (or more) genomic regions communicate with each other and at least one of them interacts with a given factor of interest, ligation will produce molecules consisting of the reporter complex nucleic acid and fragments from the two (or more) regions communicating with each other (illustrated in FIG. 5).

The PCR products generated in example A1, A2, A3 and A4 would thererefore constitute a template for amplifications with primers specific for ligation products of two different genomic sequences that are suspected to interact with each other.

These analyses will be possible provided that:
a) the reporter complex binding part is monomeric
b) the amplification products generated in A1, A2, A3, and A4 are separated from their templates before proceeding to the amplification with the above described primers specific for the ligation products of the two different genomic sequences suspected to interact. This step is necessary to avoid the amplification of those ligation products between the two interacting genomic fragments that do not involve the presence of the factor under investigation

1-12. (canceled)

13. A method of localizing a binding protein or binding site on a sample nucleic acid, comprising
(A) bringing a reporter complex, comprising at least one binding part showing specific binding to the molecule or the site of interest and at least one reporter nucleic acid, into contact with the sample nucleic acid;
(B) fragmenting the sample nucleic acid;
(C) enzymatically ligating the reporter complex nucleic acid to the sample nucleic acid; and
(D) detecting the hybrid ligation product.

14. The method of claim 13, further comprising bringing a second reporter complex, comprising at least one binding part showing specific binding to a second molecule or site of interest and at least one reporter nucleic acid, into contact with the sample nucleic acid, enzymatically ligating the second reporter complex nucleic acid to the sample nucleic acid, such that said hybrid ligation product is a double ligation product.

15. The method of claim 13, wherein the fragmentation is performed by sonication or by digestion with at least one restriction enzyme or Rnase H.

16. The method of claim 13, wherein the detection of the hybrid ligation product is performed by PCR, RCA, amplification of circularizable probes, in vitro transcription, hybridization with labelled complementary sequence probes or sequence analysis of the amplified nucleic acids.

17. The method of claim 13, wherein the reporter complex binding part is a protein.

18. A method for producing a library of fragments of interest from a sample nucleic acid, comprising
(A) bringing at least one reporter complex, comprising at least one binding part showing specific binding to a nucleic acid binding factor or a site of interest in the sample nucleic acid, and at least one reporter nucleic acid, into contact with the sample nucleic acid;
(B) fragmenting the sample nucleic acid;
(C) enzymatically ligating the reporter nucleic acid to the sample nucleic acid; and
(D) amplifying the ligated reporter/ sample nucleic acid.

19. The method of claim 18, wherein the fragmentation is performed by sonication or by digestion with at least one restriction enzyme or RnaseH.

20. The method of claim 18, wherein the reporter complex binding part is a protein.

21. A library produced by the method of claim 18.

22. The library of claim 21, wherein said library is immobilized on a solid support.

23. The library of claim 22, wherein said solid support is a microarray.

24. A solid support having immobilized thereon at least one library according to claim 21.

25. The solid support of claim 24, wherein the solid support is a microarray with each microdot comprising a library according to claim 21.

26. A kit for localizing a binding protein or binding site on a sample nucleic acid, comprising
(A) at least one reporter complex comprising a protein part showing specific binding to a molecule or a site of interest and at least one reporter nucleic acid;
(B) at least one ligase; and
(C) operating instructions.
